(19) United States
(12) Patent Application Publication
(10) Pub. No.: US 2026/0157417 A1
(43) Pub. Date: Jun. 11, 2026

(54) BOTANICAL-CONTAINING GRANULES FOR HEATED INHALABLE PRODUCTS AND METHODS OF FORMING AND USING THE SAME

(71) Applicant: Altria Client Services LLC, Richmond, VA (US)

(72) Inventors: Sarah HUYNH, Fredericksburg, VA (US); Shaoyong YU, Glen Allen, VA (US); Raymond W. LAU, Glen Allen, VA (US); Lionel DELOACH, Moseley, VA (US); Alex JORDAN, Midlothian, VA (US); Gaurav PAREKH, Richmond, VA (US); Shuzhong ZHUANG, Glen Allen, VA (US); Eric HAWES, Midlothian, VA (US); Benjamin LEWIS, Richmond, VA (US); Jesse FREDERICK, Richmond, VA (US)

(73) Assignee: Altria Client Services LLC, Richmond, VA (US)

(21) Appl. No.: 19/411,547

(22) Filed: Dec. 8, 2025

Related U.S. Application Data
(60) Provisional application No. 63/729,534, filed on Dec. 9, 2024.

(51) Int. Cl. A24B 15/16; A24B 15/28; A24B 15/32; A24B 15/38; A24B 15/40; A24D 1/18; A24D 1/20; A61K 31/465

(52) U.S. Cl. CPC A24B 15/16; A24B 15/285; A24B 15/32; A24B 15/385; A24B 15/403; A24D 1/18; A24D 1/20; A61K 31/465

(57) ABSTRACT

A method for preparing botanical granules for use in heated inhalable products may include spheronizing a wet mass to form the botanical granules, where the wet mass includes a botanical powder and a solvent, and the botanical powder includes botanical particles having average particle sizes of less than or equal to about 2 millimeters. The wet mass may be included in a granulation solution, and the method may include preparing the granulation solution by contacting the solvent to the botanical powder. The granulation solution may also further include a binder, an active material, a humectant, a controlled delivery agent, an aerosol-forming agent, or any combination thereof.

### CROSS-REFERENCE TO RELATED APPLICATION

This application claims the benefit of U.S. Provisional Application No. 63/729,534, filed on Dec. 9, 2024. The entire disclosure of the above application is incorporated herein by reference.

### BACKGROUND

### Field

The present disclosure relates to aerosol-generating substrates and consumables including the same, such as for use in aerosol-generating devices, configured to generate an aerosol without involving a substantial pyrolysis of an aerosol-forming substrate.

### Description of Related Art

Some electronic devices are configured to heat an aerosol-forming substrate, such as a plant material, to a temperature that is sufficient to release constituents of the plant material while keeping the temperature below a combustion point of the plant material so as to avoid any substantial pyrolysis of the plant material. Such devices may be referred to as aerosol-generating devices (e.g., heated tobacco aerosol-generating devices or heated tobacco product aerosol-generating devices), and the plant material heated may include tobacco, cannabis, or a combination of tobacco and cannabis. In some instances, the plant material may be introduced directly into a heating chamber of an aerosol-generating device. In other instances, the plant material may be prepackaged in individual containers to facilitate insertion and removal from an aerosol-generating device.

### SUMMARY

According to at least one example embodiment, an example method for preparing botanical granules for use in heated inhalable products is provided.

In at least one example embodiment, the method may include spheronizing a wet mass to form the botanical granules, where the wet mass includes a botanical powder and a solvent, and the botanical powder includes botanical particles having average particle sizes of less than or equal to about 2 millimeters.

In at least one example embodiment, the method may further include contacting the botanical powder with the solvent to form the wet mass.

In at least one example embodiment, the contacting may include adding the solvent to the botanical powder in increments.

In at least one example embodiment, the contacting may include applying a mixing force to the botanical powder.

In at least one example embodiment, the mixing force may be applied for a time period less than or equal to about 15 minutes.

In at least one example embodiment, the mixing force may be applied for a time period greater than or equal to about 5 minutes.

In at least one example embodiment, the method may further include, before the contacting, isolating the botanical particles having the average particle sizes of less than or equal to 2 millimeters.

In at least one example embodiment, the isolating may include moving precursor botanical particles through a mesh.

In at least one example embodiment, the solvent may include an aqueous solvent.

In at least one example embodiment, the wet mass may be included in a granulation solution.

In at least one example embodiment, the granulation solution may further include a binder, an active material, a humectant, or any combination thereof.

In at least one example embodiment, the binder may include hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

In at least one example embodiment, the active material may include nicotine.

In at least one example embodiment, the nicotine may include free base nicotine, a nicotine salt, or a combination of the free base nicotine and the nicotine salt.

In at least one example embodiment, the nicotine salt may include citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof.

In at least one example embodiment, the humectant may include 1,2-propylene glycol, 1,3-propylene glycol, 1,3-butane diol, glycerol, or any combination thereof.

In at least one example embodiment, the method may further include contacting the binder, the active material, the humectant, or the combination thereof with the wet mass to form the granulation solution.

In at least one example embodiment, the granulation solution may further include a controlled delivery agent.

In at least one example embodiment, the controlled delivery agent may include a binder.

In at least one example embodiment, the binder may include hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

In at least one example embodiment, the method may further include contacting the controlled delivery agent with the wet mass to form the granulation solution.

In at least one example embodiment, the granulation solution may further include an aerosol-forming agent.

In at least one example embodiment, the aerosol-forming agent may include 1,2-propylene glycol, 1,3-propylene glycol, 1,3-butane diol, glycerol, or any combination thereof.

In at least one example embodiment, the method may further include contacting the aerosol-forming agent with the wet mass to form the granulation solution.

In at least one example embodiment, the spheronizing may include introducing the wet mass into a container and applying a centrifugal force.

In at least one example embodiment, the container may include a stationary container.

In at least one example embodiment, the stationary container may include a friction plate.

In at least one example embodiment, a centrifugal speed of the friction plate may be greater than or equal to about 600 rotations per minute.

In at least one example embodiment, a centrifugal speed of the friction plate may be less than or equal to about 2,000 rotations per minute.

In at least one example embodiment, a centrifugal speed of the friction plate may be about 1,500 rotations per minute.

In at least one example embodiment, the friction plate may have a groove.

In at least one example embodiment, the groove may have a groove size greater than or equal to about 2 millimeters.

In at least one example embodiment, the groove may have a groove size less than or equal to about 4 millimeters.

In at least one example embodiment, the botanical granules may have an average particle size greater than or equal to 500 micrometers.

In at least one example embodiment, the botanical granules may have an average particle size less than or equal to about 2,500 micrometers.

In at least one example embodiment, the botanical granules may have a bulk density greater than or equal to about 0.23 g/cc.

In at least one example embodiment, the botanical granules may have a bulk density less than or equal to about 0.25 g/cc.

In at least one example embodiment, the botanical granules may have a tapped density greater than or equal to about 0.3 g/cc.

In at least one example embodiment, the botanical granules may have a tapped density less than or equal to about 0.32 g/cc.

In at least one example embodiment, the method may further include drying the botanical granules to remove the solvent.

In at least one example embodiment, the drying may include transferring the botanical granules to a fluidized bed.

In at least one example embodiment, the method may further include sieving the botanical granules to confirm particle size.

In at least one example embodiment, the botanical powder may include star anise, coco, fennel, eucalyptus, dill, cinnamon, fenugreek, ginger, basil, holy basil, caraway, rosemary, clove, rose petals, ginger, lavender, oak wood, or any combination thereof.

According to at least one example embodiment, another example method for preparing botanical granules for use in heated inhalable products is provided.

In at least one example embodiment, the method may include preparing a granulation solution and spheronizing the granulation solution. The granulation solution may include a botanical powder and a solvent. The botanical powder may include botanical particles having average particle sizes of less than or equal to about 2 millimeters.

In at least one example embodiment, the preparation of the granulation solution may include contacting the botanical powder to the solvent.

In at least one example embodiment, the contacting may include adding the solvent to the botanical powder in increments.

In at least one example embodiment, the contacting may include applying a mixing force to the botanical powder.

In at least one example embodiment, the mixing force may be applied for a time period greater than or equal to about 5 minutes to less than or equal to about 15 minutes.

In at least one example embodiment, the method may further include, before the preparation of the granulation solution, isolating the botanical particles having average particle sizes of less than or equal to 2 millimeters.

In at least one example embodiment, the isolating may include moving precursor botanical particles through a mesh.

In at least one example embodiment, the solvent may include an aqueous solvent.

In at least one example embodiment, the granulation solution may further include a binder, an active material, a humectant, or any combination thereof.

In at least one example embodiment, the binder may include hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

In at least one example embodiment, the active material may include nicotine.

In at least one example embodiment, the nicotine may include free base nicotine, a nicotine salt, or a combination of the free base nicotine and the nicotine salt.

In at least one example embodiment, the nicotine salt may include citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof.

In at least one example embodiment, the humectant may include 1,2-propylene glycol, 1,3-propylene glycol, 1,3-butane diol, glycerol, or any combination thereof.

In at least one example embodiment, the granulation solution may further include a controlled delivery agent.

In at least one example embodiment, the controlled delivery agent may include a binder.

In at least one example embodiment, the binder may include hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

In at least one example embodiment, the granulation solution may further include an aerosol-forming agent.

In at least one example embodiment, the aerosol-forming agent may include 1,2-propylene glycol, 1,3-propylene glycol, 1,3-butane diol, glycerol, or any combination thereof.

In at least one example embodiment, the spheronizing may include introducing the wet mass into a container and applying a centrifugal force.

In at least one example embodiment, the container may include a stationary container.

In at least one example embodiment, the stationary container may include a friction plate.

In at least one example embodiment, the centrifugal speed of the friction plate may be greater than or equal to about 600 rotations per minute to less than or equal to about 2,000 rotations per minute.

In at least one example embodiment, the friction plate may have a groove.

In at least one example embodiment, the groove may have a groove size greater than or equal to about 2 millimeters to less than or equal to about 4 millimeters.

In at least one example embodiment, the botanical granules may have an average particle size greater than or equal to 500 micrometers to less than or equal to about 2,500 micrometers.

In at least one example embodiment, the botanical granules may have a bulk density greater than or equal to about 0.23 g/cc to less than or equal to about 0.25 g/cc.

In at least one example embodiment, the botanical granules may have a tapped density greater than or equal to about 0.3 g/cc to less than or equal to about 0.32 g/cc.

In at least one example embodiment, the method may further include drying the botanical granules to remove the solvent.

In at least one example embodiment, the drying may include transferring the botanical granules to a fluidized bed.

In at least one example embodiment, the method may further include sieving the botanical granules to confirm particle size.

In at least one example embodiment, the botanical powder may include star anise, coco, fennel, eucalyptus, dill, cinnamon, fenugreek, ginger, basil, holy basil, caraway, rosemary, clove, rose petals, ginger, lavender, oak wood, or any combination thereof.

According to at least one example embodiment, a heated inhalable product is provided.

In at least one example embodiment, the heated inhalable product may include a device body that includes a housing that defines a chamber, a heater, and a power supply that is configured to supply power to the heater. The heated inhalable product may also include a consumable configured to be at least partially received in the chamber. The consumable may include a container defining an annular interior region, where an outer surface of the container configured to be heated by the heater. The consumable may also include a filler in the annular interior region, where the filler includes substantially homogeneous botanical particles.

In at least one example embodiment, the annular interior region may further include a binder, an active material, a humectant, or any combination thereof.

In at least one example embodiment, the binder may include hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

In at least one example embodiment, the active material may include nicotine.

In at least one example embodiment, the nicotine may include free base nicotine, a nicotine salt, or a combination of the free base nicotine and the nicotine salt.

In at least one example embodiment, the nicotine salt may include citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof.

In at least one example embodiment, the humectant may include 1,2-propylene glycol, 1,3-propylene glycol, 1,3-butane diol, glycerol, or any combination thereof.

In at least one example embodiment, the annular interior region may further include a controlled delivery agent.

In at least one example embodiment, the controlled delivery agent may include a binder.

In at least one example embodiment, the binder may include hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

In at least one example embodiment, the annular interior region may further include an aerosol-forming agent.

In at least one example embodiment, the aerosol-forming agent may include 1,2-propylene glycol, 1,3-propylene glycol, 1,3-butane diol, glycerol, or any combination thereof.

In at least one example embodiment, the botanical particles may be prepared by spheronizing a wet mass including precursor botanical particles, the precursor botanical particles having average particle sizes of less than or equal to about 2 millimeters.

According to at least one example embodiment, another example method for preparing botanical granules for use in heated inhalable products is provided.

In at least one example embodiment, the method may include spheronizing a wet mass to form the botanical granules, where the wet mass includes a botanical powder and a solvent. The botanical powder may include botanical particles having average particle sizes of less than or equal to about 2 millimeters.

In at least one example embodiment, the method may further include contacting the botanical powder with the solvent to form the wet mass. The contacting may include at least one of adding the solvent to the botanical powder in increments and applying a mixing force to the botanical powder.

In at least one example embodiment, the method may further include, before the contacting, isolating the botanical particles having the average particle sizes of less than or equal to 2 millimeters.

In at least one example embodiment, the solvent may be an aqueous solvent.

In at least one example embodiment, the wet mass may be included in a granulation solution and the granulation solution may further include a binder, an active material, a humectant, or any combination thereof.

In at least one example embodiment, the binder may include hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof; the active material may include nicotine; and the humectant may include 1,2-propylene glycol, 1,3-propylene glycol, 1,3-butane diol, glycerol, or any combination thereof.

In at least one example embodiment, the method may further include contacting the binder, the active material, the humectant, or the combination thereof with the wet mass to form the granulation solution.

In at least one example embodiment, the granulation solution may further include an aerosol-forming agent.

In at least one example embodiment, the aerosol-forming agent may include 1,2-propylene glycol, 1,3-propylene glycol, 1,3-butane diol, glycerol, or any combination thereof.

In at least one example embodiment, the method may further include contacting the aerosol-forming agent with the wet mass to form the granulation solution.

In at least one example embodiment, the spheronizing may include introducing the wet mass into a stationary bowl including a friction plate; and applying a centrifugal force.

In at least one example embodiment, a centrifugal speed of the friction plate may be greater than or equal to about 600 rotations per minute to less than or equal to about 2,000 rotations per minute.

In at least one example embodiment, the friction plate may have a groove.

In at least one example embodiment, the groove may have a groove size greater than or equal to about 2 millimeters to less than or equal to about 4 millimeters.

In at least one example embodiment, the botanical granules may have an average particle size greater than or equal to 500 micrometers to less than or equal to about 2,500 micrometers. The botanical granules may have a bulk density greater than or equal to about 0.23 g/cc to less than or equal to about 0.25 g/cc. The botanical granules may have a tapped density greater than or equal to about 0.3 g/cc to less than or equal to about 0.32 g/cc.

In at least one example embodiment, the method may further include drying the botanical granules to remove the solvent by transferring the botanical granules to a fluidized bed.

In at least one example embodiment, the method may further include sieving the botanical granules to confirm particle size.

According to at least one example embodiment, another example method for preparing botanical granules for use in heated inhalable products is provided.

In at least one example embodiment, the method may include preparing a granulation solution, where the granulation solution includes a botanical powder and a solvent, and spheronizing the granulation solution. The botanical powder may include botanical particles having average particle sizes of less than or equal or equal to about 2 millimeters.

In at least one example embodiment, the preparation of the granulation solution may include contacting the botanical powder to the solvent, where the contacting includes at least one of adding the solvent to the botanical powder in increments and applying a mixing force to the botanical powder.

In at least one example embodiment, the spheronizing may include introducing the granulation solution into a stationary bowl including a friction plate; and applying a centrifugal force.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a perspective view of an example aerosol-forming substrate in loose form according to at least one example embodiment.

FIG. 2 is a perspective view of an example consumable including an aerosol-forming substrate (like the aerosol-forming substrate illustrated in FIG. 1) according to at least one example embodiment.

FIG. 3 is a cross-sectional view of the consumable illustrated in FIG. 2 taken along line III-III according to at least one example embodiment.

FIG. 4 is a perspective view of the consumable as illustrated in FIG. 2 including an external heater according to at least one example embodiment.

FIG. 5 is a perspective view of an aerosol-generating device including a consumable (like the consumable illustrated in FIG. 2) and a heater (like the heater illustrated in FIG. 4) according to at least one example embodiment.

FIG. 6 is a schematic diagram of the aerosol-generating device as illustrated in FIG. 5 according to at least one example embodiment.

FIG. 7 is a flowchart illustrating an example method of preparing an aerosol-forming substrate (like the aerosol-forming substrate illustrated in FIG. 1) according to at least one example embodiment.

FIG. 8 is a flowchart illustrating another example method of preparing an aerosol-forming substrate (like the aerosol-forming substrate illustrated in FIG. 1) according to at least one example embodiment.

FIG. 9 is a flowchart illustrating another example method of preparing an aerosol-forming substrate (like the aerosol-forming substrate illustrated in FIG. 1) according to at least one example embodiment.

FIG. 10 is a flowchart illustrating another example method of preparing an aerosol-forming substrate (like the aerosol-forming substrate illustrated in FIG. 1) according to at least one example embodiment.

### DETAILED DESCRIPTION OF EXAMPLE EMBODIMENTS

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives thereof. Like numbers refer to like elements throughout the description of the figures.

It should be understood that when an element or layer is referred to as being “on,” “connected to,” “coupled to,” “attached to,” “adjacent to,” or “covering” another element or layer, it may be directly on, connected to, coupled to, attached to, adjacent to or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations or sub-combinations of one or more of the associated listed items.

It should be understood that, although the terms first, second, third, etc. may be used herein to describe various elements, components, regions, layers and/or sections, these elements, components, regions, layers, and/or sections should not be limited by these terms. These terms are only used to distinguish one element, component, region, layer, or section from another region, layer, or section. Thus, a first element, component, region, layer, or section discussed below could be termed a second element, component, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

While the term “same” or “identical” is used in description of example embodiments, it should be understood that some imprecisions may exist. Thus, when one element is referred to as being the same as another element, it should be understood that an element or a value is the same as another element within a desired manufacturing or operational tolerance range (e.g., ±10%).

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes.

The controller may include processing circuitry such as hardware including logic circuits; a hardware/software combination such as a processor executing software stored in a memory; or a combination thereof. For example, the processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

Unless otherwise defined, all terms (including technical and scientific terms) used herein have the same meaning as commonly understood by one of ordinary skill in the art to which example embodiments belong. It will be further understood that terms, including those defined in-commonly used dictionaries, should be interpreted as having a meaning that is consistent with their meaning in the context of the relevant art and will not be interpreted in an idealized or overly formal sense unless expressly so defined herein.

Some electronic devices are configured to heat a material (e.g., plant material) to a temperature that is sufficient to release constituents of the material while keeping the temperature below its ignition temperature so as to avoid a self-sustaining burning or a self-sustaining combustion of the material (i.e., in contrast to where a material is lit, such as lit-end cigarettes). Such devices may be characterized as generating an aerosol of constituents released by heating and may be referred to as heated products, heated non-tobacco products, or heated tobacco products.

It is understood that heating of a material below its ignition temperature may, in some circumstances, produce incidental and insubstantial levels of oxidized or other thermal decomposition byproducts. However, in some example embodiments, the heating in aerosol-generating devices may be below the pyrolysis temperature of the material so as to produce an aerosol having no or insubstantial levels of thermal decomposition byproducts of the material. Thus, in at least one example embodiment, pyrolysis of the material may not occur during the heating and resulting production of aerosol. In other example embodiments, there may be incidental pyrolysis, with production of oxidized or other thermal decomposition byproducts at levels that are insignificant relative to the primary constituents released by heating of the material.

An aerosol-forming substrate includes a material or combination of materials that may yield an aerosol. An aerosol relates to the matter generated or output by the devices disclosed, claimed, and equivalents thereof. The material may include a compound (e.g., nicotine, cannabinoid, cannabimimetic agent) that is released when the material is heated. In such instances, an aerosol including the compound may be produced when the material is heated. The heating may be below the ignition temperature so as to avoid a self-sustaining burning or a self-sustaining combustion of the material (i.e., in contrast to where a material is lit, such as lit-end cigarettes). It is understood that heating of a material below its ignition temperature may, in some circumstances, produce incidental and insubstantial levels of oxidized or other thermal decomposition byproducts. However, in some example embodiments, the heating in aerosol-generating devices may be below the pyrolysis temperature of the material so as to produce an aerosol having no or insubstantial levels of thermal decomposition byproducts of the material. Thus, in at least one example embodiment, pyrolysis of the material may not occur during the heating and resulting production of aerosol. In other example embodiment, there may be incidental pyrolysis, with production of oxidized or other thermal decomposition byproducts at levels that are insignificant relative to the primary constituents released by heating of the material.

The material(s) of the aerosol-forming substrate may include a fibrous material. For instance, the fibrous material may include a botanical material. The fibrous material may be configured to release a compound when heated. The compound may be a naturally occurring constituent of the fibrous material. For instance, the fibrous material may include a plant material, such as tobacco, and the compound released may include nicotine. The term “tobacco” includes any tobacco plant material, such as tobacco leaf, tobacco plug, reconstituted tobacco, compressed tobacco, shaped tobacco, powder tobacco, or any combination thereof, from one or more species of tobacco plants, including, for example, Nicotiana rustica, Nicotiana tabacum, or a combination of Nicotiana rustica and Nicotiana tabacum.

In some example embodiments, the tobacco material may include material from any member of the genus Nicotiana. In some example embodiments, the tobacco material may include a blend of two or more different tobacco varieties. Tobacco materials that may be used include, for example, flue-cured tobacco, Burley tobacco, Dark tobacco, Maryland tobacco, Oriental tobacco, rare tobacco, specialty tobacco, the like, or any combination thereof. The tobacco material may be provided in any suitable form, including, for example, tobacco lamina, processed tobacco materials (such as volume expanded or puffed tobacco), processed tobacco stems (such as cut-rolled or cut-puffed stems), reconstituted tobacco materials, the like, or any combination thereof. In some example embodiments, the tobacco material may be in the form of a substantially dry tobacco mass. In some example embodiments, the tobacco material may be mixed and/or combined with propylene glycol, glycerin, or a combination of propylene glycol and glycerin.

The compound in the generated aerosol may also be a naturally occurring constituent of a medicinal plant that has a medically-accepted physiological effect (e.g., therapeutic effect, prophylactic effect). In some example embodiments, the medicinal plant may include a cannabis plant, a cannabimimetic plant (i.e., a plant with similar pharmacological effects to those of cannabis), or a combination of a cannabis plant and a cannabimimetic plant. In the instance of a cannabis plant, the compound may include a cannabinoid. Cannabinoids may interact with receptors in the body to produce a wide range of effects. As a result, cannabinoids have been used for a variety of medicinal purposes (e.g., treatment of pain, nausea, epilepsy, psychiatric disorders). The fibrous material may include leaf material, flower material, or the combination of the leaf material and the flower material from one or more species of cannabis plants, such as Cannabis sativa, Cannabis indica, Cannabis ruderalis, or any combination thereof. In at least one example embodiment, the fibrous material may include an amount of Cannabis sativa greater than or equal to about 60 weight percent to less than or equal to about 80 weight percent (e.g., about 70 weight percent) and greater than or equal to about 20 weight percent to less than or equal to about 40 weight % (e.g., about 30 weight percent) of Cannabis indica. For a cannabimimetic plant, the compound may include a cannabimimetic agent. Cannabimimetic agents may interact with receptors in the body to produce similar pharmacological effects as cannabinoids.

Cannabinoids may include, for example, tetrahydrocannabinolic acid (THCA), tetrahydrocannabinol (THC), cannabidiolic acid (CBDA), cannabidiol (CBD), cannabinol (CBN), cannabicyclol (CBL), cannabichromene (CBC), cannabigerol (CBG), or any combination thereof. Tetrahydrocannabinolic acid (THCA) may be a precursor of tetrahydrocannabinol (THC), while cannabidiolic acid (CBDA) may be precursor of cannabidiol (CBD). Tetrahydrocannabinolic acid (THCA) and cannabidiolic acid (CBDA) may be converted to tetrahydrocannabinol (THC) and cannabidiol (CBD), respectively, via heating. In at least one example embodiment, heat from a heater may cause decarboxylation so as to convert the tetrahydrocannabinolic acid (THCA) to tetrahydrocannabinol (THC) and/or to convert the cannabidiolic acid (CBDA) to cannabidiol (CBD).

In instances where both tetrahydrocannabinolic acid (THCA) and tetrahydrocannabinol (THC) are present, the decarboxylation and resulting conversion may cause a decrease in tetrahydrocannabinolic acid (THCA) and an increase in tetrahydrocannabinol (THC). For example, in at least one example embodiment, at least about 50 % (e.g., at least about 87 %) of the tetrahydrocannabinolic acid (THCA) may be converted to tetrahydrocannabinol (THC) during the heating. In instances where both cannabidiolic acid (CBDA) and cannabidiol (CBD) are present, the decarboxylation and resulting conversion may cause a decrease in cannabidiolic acid (CBDA) and an increase in cannabidiol (CBD). For example, in at least one example embodiment, at least about 50 % (e.g., at least about 87 %) of the cannabidiolic acid (CBDA) may be converted to cannabidiol (CBD) during the heating.

The compound that is released may be or may additionally include a non-naturally occurring additive that is subsequently introduced into the fibrous material. In at least one example embodiment, the fibrous material may include cotton, polyethylene, polyester, rayon, the like (e.g., in a form of a gauze), or any combination thereof. In at least one example embodiment, the fibrous material may include a cellulose material (e.g., non-tobacco and/or non-cannabis material). In each instance, the compound introduced may include nicotine, cannabinoids, cannabimimetic agents, flavorants, or any combination thereof. The flavorants may be from natural sources, including, for example, plant extracts (e.g., tobacco extract, cannabis extract, cannabimimetic extract) and/or artificial sources. In at least one example embodiment, when the fibrous material includes tobacco, cannabis, or a combination of tobacco and cannabis, the compound may be, or may additionally include, one or more flavorants (e.g., menthol, mint, vanilla). For example, the compound within the aerosol-forming substrate may include naturally occurring constituents, non-naturally occurring additives, or a combination of naturally occurring constituents and non-naturally occurring additives. In at least one example embodiment, levels of the naturally occurring constituents of the aerosol-forming substrate may be increased through supplementation. For example, the existing levels of nicotine in a quantity of tobacco may be increased through supplementation with an extract containing nicotine. Similarly, the existing levels of one or more cannabinoids in a quantity of cannabis may be increased through supplementation with an extract containing such cannabinoids. Likewise, the existing levels of one or more cannabimimetic agents in a quantity of cannabimimetic material may be increased through supplementation with an extract containing such cannabimimetic agents.

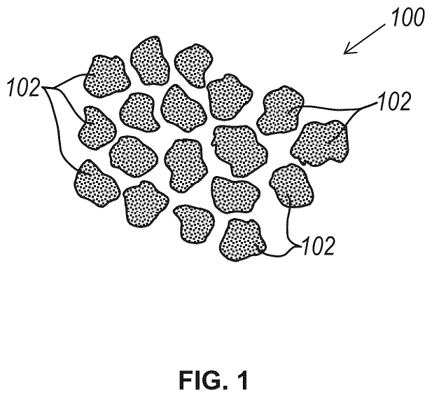

FIG. 1 is a perspective view of an example aerosol-forming substrate 100 in loose form according to at least one example embodiment.

The aerosol-forming substrate (which may also be referred to as a “substrate” or “filler”) 100 may include a plurality of granules 102. In at least one example embodiment, each of the plurality of granules 102 may have a substantially spherical shape. In at least one example embodiment, each of the plurality of granules 102 may have an average particle size greater than or equal to about 0.2 millimeters (e.g., greater than or equal to about 0.3 millimeters, greater than or equal to about 0.4 millimeters, greater than or equal to about 0.5 millimeters, greater than or equal to about 0.75 millimeters, greater than or equal to about 1 millimeters, greater than or equal to about 1.25 millimeters, greater than or equal to about 1.5 millimeters, greater than or equal to about 1.75 millimeters, greater than or equal to about 2 millimeters, or greater than or equal to about 2.25 millimeters). In at least one example embodiment, the average size of the granules 102 may be less than or equal to about 2.5 millimeters (e.g., less than or equal to about 2.25 millimeters, less than or equal to about 2 millimeters, less than or equal to about 1.75 millimeters, less than or equal to about 1.5 millimeters, less than or equal to about 1.25 millimeters, less than or equal to about 1 millimeters, less than or equal to about 0.75 millimeters, less than or equal to about 0.5 millimeters, less than or equal to about 0.4 millimeters, or less than or equal to about 0.3 millimeters). In at least one example embodiment, each of the plurality of granules 102 may have an average particle size greater than or equal to about 0.2 millimeters to less than or equal to about 2.5 millimeters.

Each of the plurality of granules 102 may include a plurality of particles. The plurality of particles may include a fibrous material, a botanical material, or a combination of fibrous and botanical materials. In each instance, the fibrous material and the botanical material may be configured to release one or more constituents or compounds when heated. In at least one example embodiment, the botanical material may include, for example, star anise, coco, fennel, eucalyptus, dill, cinnamon, fenugreek, ginger, basil, holy basil, caraway, rosemary, clove, rose petals, ginger, lavender, oak wood, or any combination thereof.

In at least one example embodiment, the plurality of particles may have an average particle size of greater than or equal to about 500 micrometers (e.g., greater than or equal to about 600 micrometers, greater than or equal to about 700 micrometers, greater than or equal to about 800 micrometers, greater than or equal to about 900 micrometers, greater than or equal to about 1,000 micrometers, greater than or equal to about 1,100 micrometers, greater than or equal to about 1,200 micrometers, greater than or equal to about 1,300 micrometers, greater than or equal to about 1,400 micrometers, greater than or equal to about 1,500 micrometers, greater than or equal to about 1,600 micrometers, greater than or equal to about 1,700 micrometers, greater than or equal to about 1,800 micrometers, greater than or equal to about 1,900 micrometers, greater than or equal to about 2,000 micrometers, greater than or equal to about 2,100 micrometers, greater than or equal to about 2,200 micrometers, greater than or equal to about 2,300 micrometers, or greater than or equal to about 2,400 micrometers). In at least one example embodiment, the plurality of particles may have an average size of less than or equal to about 2,500 micrometers (e.g., less than or equal to about 2,400 micrometers, less than or equal to about 2,300 micrometers, less than or equal to about 2,200 micrometers, less than or equal to about 2,100 micrometers, less than or equal to about 2,000 micrometers, less than or equal to about 1,900 micrometers, less than or equal to about 1,800 micrometers, less than or equal to about 1,700 micrometers, less than or equal to about 1,600 micrometers, less than or equal to about 1,500 micrometers, less than or equal to about 1,400 micrometers, less than or equal to about 1,300 micrometers, less than or equal to about 1,200 micrometers, less than or equal to about 1,100 micrometers, less than or equal to about 1,000 micrometers, less than or equal to about 900 micrometers, less than or equal to about 800 micrometers, less than or equal to about 700 micrometers, or less than or equal to about 600 micrometers). In at least one example embodiment, the plurality of particles may have an average particle size of greater than or equal to about 500 micrometers to less than or equal to about 2,500 micrometers.

One or more of the plurality of granules 102 may also further include a binder, an active material, a humectant, a controlled delivery agent, an aerosol-forming agent (which may also be referred to as an “aerosol former”), or any combination thereof.

In at least one example embodiment, one or more of the plurality of granules 102 may include a binder. The binder may at least partially surround and/or encapsulate the individual particles and/or the collection of particles defining the individual granules 102. The binder may be selected to hold together the plurality of particles to define the individual granules 102. In at least one example embodiment, the binder may include, for example, hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof. In at least one example embodiment, the binder may include, for example, cellulose, microcrystalline cellulose (MCC), hydroxypropylcellulose (HPC), methylcellulose (MC), ethyl cellulose (EC), hydroxypropylmethylcellulose (HPMC), sodium carboxymethylcellulose (NaCMC), starch, pregelatinized starch, gum acacia, maltodextrin, povidone (PVP), polyvinyl alcohol (PVA), polyvinylethylene glycol (PEG), polyvinyl alcohol graft polyethylene glycol copolymer (PVA-g-PEG), polymethacrylate, a sugar alcohol, or any combination thereof. The sugar alcohol may include, for example, ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof.

In at least one example embodiment, each of the plurality of granules 102 may include greater than or equal to 0 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, greater than or equal to about 4.5 weight percent, or greater than or equal to about 5 weight percent) of the binder. In at least one example embodiment, each of the plurality of granules 102 may include less than or equal to about 5.4 weight percent (e.g., less than or equal to about 5 weight percent, less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent) of the binder. In at least one example embodiment, each of the plurality of granules 102 may include greater than or equal to 0 weight percent to less than or equal to about 5.4 weight percent of the binder.

In at least one example embodiment, one or more of the plurality of granules 102 may include an active material. The active material may at least partially surround and/or encapsulate the individual particles and/or the collection of particles defining the individual granules 102. The active material may include, for example, nicotine. The nicotine may include free base nicotine, a nicotine salt, or a combination of the free base nicotine and the nicotine salt. The nicotine salt may include, for example, citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof.

In at least one example embodiment, each of the plurality of granules 102 may include greater than or equal to 0 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, or greater than or equal to about 3 weight percent) of the active material. In at least one example embodiment, each of the plurality of granules 102 may include less than or equal to about 3.5 weight percent (e.g., less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent) of the active material. In at least one example embodiment, each of the plurality of granules 102 may include greater than or equal to 0 weight percent to less than or equal to about 3.5 weight percent of the active material.

In at least one example embodiment, one or more of the plurality of granules 102 may include a humectant. The humectant may at least partially surround and/or encapsulate the individual particles and/or the collection of particles defining the individual granules 102. The humectant may include, for example, 1,2-propylene glycol, 1,3-propylene glycol, 1,3-butane diol, glycerol, or any combination thereof.

In at least one example embodiment, each of the plurality of granules 102 may include greater than or equal to about 25 weight percent (e.g., greater than or equal to about 26 weight percent, greater than or equal to about 27 weight percent, greater than or equal to about 28 weight percent, greater than or equal to about 29 weight percent, greater than or equal to about 30 weight percent, or greater than or equal to about 31 weight percent) of the humectant. In at least one example embodiment, each of the plurality of granules 102 may include less than or equal to about 32 weight percent (e.g., less than or equal to about 31. 5, less than or equal to about 31.1, less than or equal to about 31, less than or equal to about 30, less than or equal to about 28, less than or equal to about 27, or less than or equal to about 26) of the humectant. In at least one example embodiment, each of the plurality of granules 102 may include greater than or equal to about 25 weight percent to less than or equal to about 32 weight percent of the humectant

In at least one example embodiment, one or more of the plurality of granules 102 may include a controlled delivery agent. The controlled delivery agent may at least partially surround and/or encapsulate the individual particles and/or the collection of particles defining the individual granules 102. The controlled delivery agent may include, for example, a binder, or a combination thereof. The binder may include, for example, hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

In at least one example embodiment, each of the plurality of granules 102 may include greater than or equal to 0 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, greater than or equal to about 4.5 weight percent, or greater than or equal to about 5 weight percent) of the controlled delivery agent. In at least one example embodiment, each of the plurality of granules 102 may include less than or equal to about 5.4 weight percent (e.g., (e.g., less than or equal to about 5 weight percent, less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent) of the controlled delivery agent. In at least one example embodiment, each of the plurality of granules 102 may include greater than or equal to 0 weight percent to less than or equal to about 5.4 weight percent of the controlled delivery agent.

In at least one example embodiment, one or more of the plurality of granules 102 may include an aerosol-forming agent. The aerosol-forming agent may at least partially surround and/or encapsulate the individual particles and/or the collection of particles defining the individual granules 102. The aerosol-forming agent may include, for example, 1,2-propylene glycol, 1,3-propylene glycol, 1,3-butane diol, glycerol, or any combination thereof.

In at least one example embodiment, each of the plurality of granules 102 may include greater than or equal to about 25 weight percent (e.g., greater than or equal to about 26 weight percent, greater than or equal to about 27 weight percent, greater than or equal to about 28 weight percent, greater than or equal to about 29 weight percent, greater than or equal to about 30 weight percent, or greater than or equal to about 31 weight percent) of the aerosol-forming agent. In at least one example embodiment, each of the plurality of granules 102 may include less than or equal to about 32 weight percent (e.g., less than or equal to about 31. 5, less than or equal to about 31.1, less than or equal to about 31, less than or equal to about 30, less than or equal to about 28, less than or equal to about 27, or less than or equal to about 26) of the aerosol-forming agent. In at least one example embodiment, each of the plurality of granules 102 may include greater than or equal to about 25 weight percent to include less than or equal to about 32 weight percent of the aerosol-forming agent.

In at least one example embodiment, the aerosol-forming substrate 100 may have a bulk density greater than or equal to about 0.23 g/cc (e.g., greater than or equal to about 0.235 g/cc, greater than or equal to about 0.24 g/cc, or greater than or equal to about 0.245 g/cc). In at least one example embodiment, the aerosol-forming substrate 100 may have a bulk density less than or equal to about 0.25 g/cc (e.g., less than or equal to about 0.245 g/cc, less than or equal to about 0.24 g/cc, or less than or equal to about 0.235 g/cc). In at least one example embodiment, the aerosol-forming substrate 100 may have a bulk density greater than or equal to about 0.23 g/cc to less than or equal to about 0.25 g/cc.

In at least one example embodiment, the aerosol-forming substrate 100 may have a tapped density greater than or equal to about 0.3 g/cc (e.g., greater than or equal to about 0.305 g/cc, greater than or equal to about 0.31 g/cc, or greater than or equal to about 0.315 g/cc). In at least one example embodiment, the aerosol-forming substrate 100 may have a tapped density less than or equal to about 0.32 g/cc (e.g., less than or equal to about 0.315 g/cc, less than or equal to about 0.31 g/cc, or less than or equal to about 0.305 g/cc). In at least one example embodiment, the aerosol-forming substrate 100 may have a tapped density greater than or equal to about 0.3 g/cc to less than or equal to about 0.32 g/cc.

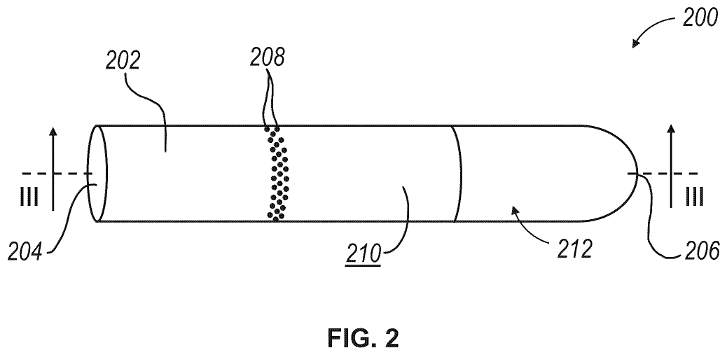

FIG. 2 is a perspective view of an example consumable 200 according to at least one example embodiment.

In at least one example embodiment, the consumable (which may also be referred to as a “capsule”) 200 may include an outer housing 202. The outer housing 202 may have a first end 204 and a second opposing end 206, where a heating region 212 is disposed nearer to the second end 206 than the first end 204.

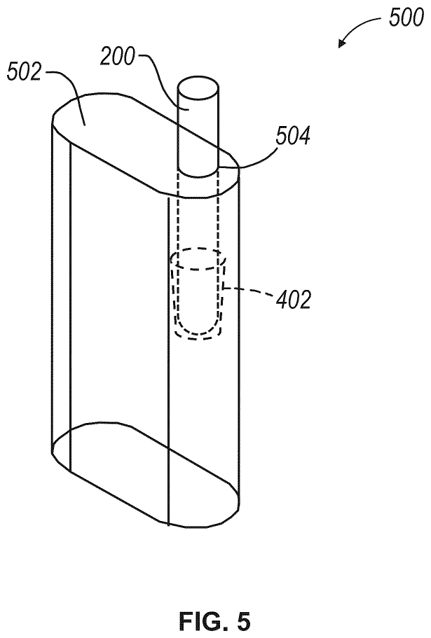

In at least one example embodiment, the first end 204 may include a substantially flat surface, and the second end 206 may include a rounded end surface. Although the outer housing 202 is illustrated as having opposing and rounded surfaces, it should be appreciated that the outer housing 202, and the consumable 200, may take a variety of configurations. In each instance, when the consumable 200 is placed within an aerosol-generating device (such as the aerosol-generating device 500 illustrated in FIG. 5), the first end 204 may be a top end, and the second end 206 is a bottom end.

The outer housing 202 defines an outer surface 210. In at least one example embodiment, the outer housing 202 may include an air inlet extending through the outer surface 210. For example, the outer housing 202 may include a first plurality of perforations 208 that define the air inlet.

In at least one example embodiment, the first plurality of perforations 208 may include, for example, greater than or equal to 2 perforations (e.g., greater than or equal to 5 perforations, greater than or equal to 10 perforations, greater than or equal to 15 perforations, greater than or equal to 20 perforations, greater than or equal to 25 perforations, greater than or equal to 30 perforations, greater than or equal to 35 perforations, greater than or equal to 40 perforations, greater than or equal to 45 perforations, greater than or equal to 50 perforations, greater than or equal to 55 perforations, greater than or equal to 60 perforations, greater than or equal to 65 perforations, greater than or equal to 70 perforations, greater than or equal to 75 perforations, greater than or equal to 80 perforations, greater than or equal to 85 perforations, greater than or equal to 90 perforations, or greater than or equal to 95 perforations). In at least one example embodiment, the first plurality of perforations 208 may include, for example, less than or equal to 100 perforations (e.g., less than or equal to 95 perforations, less than or equal to 90 perforations, less than or equal to 85 perforations, less than or equal to 80 perforations, less than or equal to 75 perforations, less than or equal to 70 perforations, less than or equal to 65 perforations, less than or equal to 60 perforations, less than or equal to 55 perforations, less than or equal to 50 perforations, less than or equal to 45 perforations, less than or equal to 40 perforations, less than or equal to 35 perforations, less than or equal to 30 perforations, less than or equal to 25 perforations, less than or equal to 20 perforations, less than or equal to 15 perforations, less than or equal to 10 perforations, or less than or equal to 5 perforations). In at least one example embodiment, the first plurality of perforations 208 may include, for example, greater than or equal to 2 perforations to less than or equal to 100 perforations.

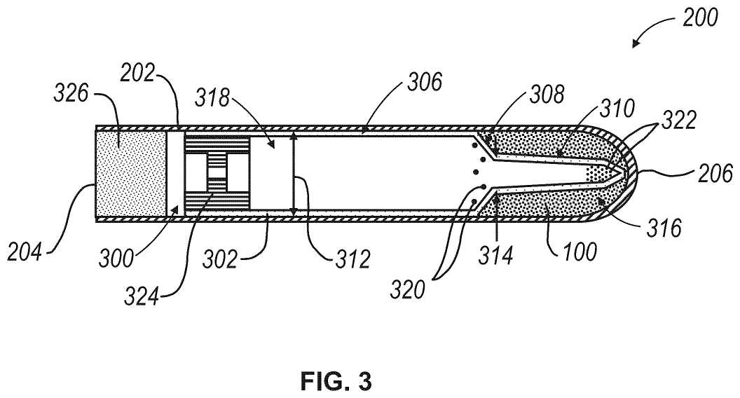

FIG. 3 is a sectional view of the consumable 200 as illustrated in FIG. 2 taken along line III-III according to at least one example embodiment.

The outer housing 202 of the consumable 200 may define a first interior region 300 and a second interior region 318. In at least one example embodiment, the consumable 200 may further include an inner housing or pod 302. The inner housing 302 may be at least partially disposed in the first interior region 300. The inner housing 302 may include a first portion 306, a second portion 308, and a third portion 310, where the second portion 308 is disposed between the first portion 306 and the third portion 310. The first portion 306 may have a first diameter 312. The third portion 310 may have a second diameter 314, at a largest point, that is smaller than the first diameter 312. The second portion 308 may have a tapered diameter joining the first diameter 312 and the second diameter 314. In at least one example embodiment, the first portion 306 may be substantially cylindrical. In at least one example embodiment, the third portion 310 may be substantially cylindrical, conical, frusto-conical, or dart-shaped.

The outer housing 202 and the inner housing 302 may cooperate to at least partially define a substrate region 316. For example, in at least one example embodiment, the second and third portions 308, 310 of the inner housing 302 may cooperate with a portion of the outer housing 202 to define the substrate region 316. In at least one example embodiment, the substrate region 316 may have a substantially annular shape. In each instance, the substrate region 316 is configured to receive a substrate. In at least one example embodiment, the substrate region 316 may be configured to receive a loosely deposited aerosol-forming substrate, like the aerosol-forming substrate 100 illustrated in FIG. 1. In other example embodiments, the substrate region 316 may be configured to receive an aerosol-forming substrate having a consolidated format.

The inner housing 302 may define a second plurality of perforations 320. For example, in at least one example embodiment, the second portion 308 of the inner housing 302 may define the second plurality of perforations 320. The second plurality of perforations 320 may provide fluid communication between the first and second interior regions 300, 318.

The inner housing 302 may define a third plurality of perforations 322. For example, in at least one example embodiment, the third portion 310 of the inner housing 302 may define the third plurality of perforations 322. The third plurality of perforations 322 may provide fluid communication between the first and second interior regions 300, 318.

The consumable 200 may include a flow restrictor 324. For example, in at least one example embodiment, the second interior region 318 of the consumable 200 may include the flow restrictor 324. The flow restrictor 324 may include one or more orifices therein.

The consumable 200 may include a filter 326. For example, in at least one example embodiment, the first interior region 300 of the consumable 200 may include the filter 326. The filter 326 may be disposed at or near the first end 204 of the outer housing 202. In at least one example embodiment, the filter 326 may include a plug of cellulose acetate tow.

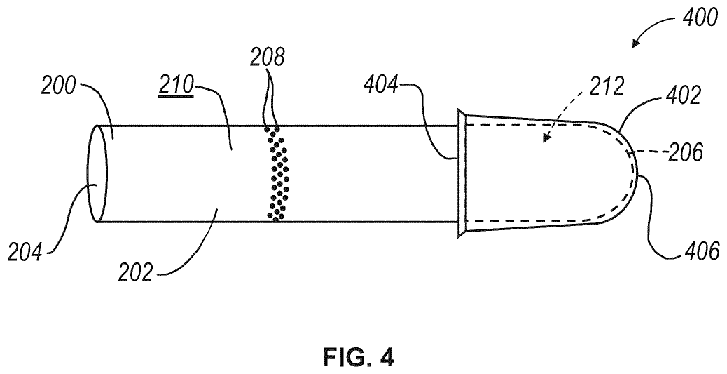

FIG. 4 is a perspective view of a consumable-heater assembly 400 that includes a consumable (like the consumable 200 as illustrated in FIG. 2) and a heater 402 according to at least one example embodiment.

The heater 402 may be an external heater that is configured to indirectly heat (for example, via heat transfer) the aerosol-forming substrate 100 supported by the consumable 200. For example, the heater 402 may be configured to apply heat to the outer surface 210 of the outer housing 202 of the consumable 200. In at least one example embodiment, the heater 402 may be configured to apply heat to the heating region 212 of the consumable 200.

In at least one example embodiment, the heater 402 may include an open end 404 and an opposing closed end 406. In this manner, the heater 402 may be configured to receive at least a portion of the consumable 200 via the open end 404. The closed end 406 may be rounded and configured to fit snugly about the received end (e.g., the second end 206) of the consumable 200. In at least one example embodiment, at least a portion of the heating region 212 of the outer surface 210 may be in direct contact with the heater 402. In other example embodiments, however, the heating region 212 of the outer surface 210 may not be direct contact with the heater 402.

FIG. 5 is a perspective view of a heated tobacco device 500 including a consumable (like the consumable 200 illustrated in FIG. 2) and a heater (like the heater 402 illustrated in FIG. 4) according to at least one example embodiment.

The device 500 may include a device body 502, where the body 502 defines an opening 504 that at least partially receives the consumable 200. In at least one example embodiment, the device 500 may be free of a distinct mouthpiece, and the consumable 200 may include a mouthpiece. Although not illustrated it should be appreciated that, in various other example embodiments, the device 500 may include a mouthpiece, and the consumable may be fully received within the device 500.

The heater 402 may be part of the device 500 or the consumable 200. In at least one example embodiment, the device 500 is configured to receive the heater 402, such that the device 500 may be free of an internal heater, such as a blade that extends into an aerosol-forming substrate 100. The heater 402 may be selected to provide uniform heating of the aerosol-forming substrate 100 as compared to an internal blade heater, which may create a radial temperature gradient within an aerosol-forming substrate. Although the heater 402 is discussed as part of the consumable 200, it should be appreciated that, in at least one other example embodiment, the heater 402 may be provided as part of the device 500.

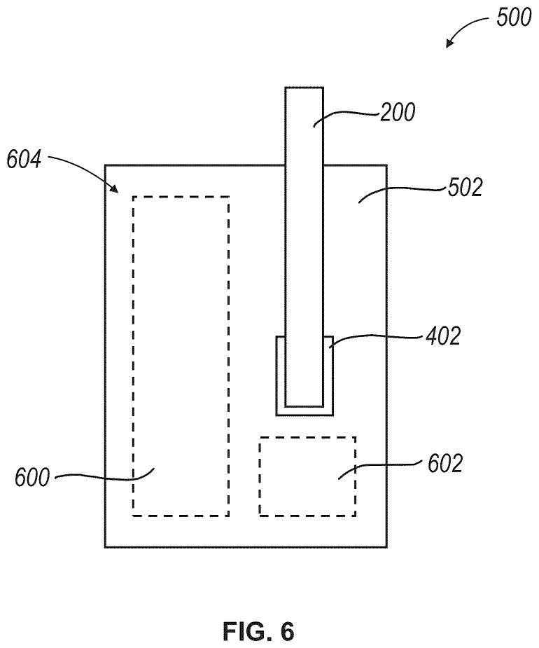

FIG. 6 is a schematic view of the aerosol-generating device 500 as illustrated in FIG. 5 according to at least one example embodiment.

The device body 502 may encase or house the heater 402, a power source 600, and a processing or control circuity 602 within an interior region or chamber 604. The control circuity 602 may be hardware including logic circuits; a hardware/software combination such as a processor executing software; or a combination thereof. For example, the control circuitry may include a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), the like, or any combination thereof. The supply of current from the power source 600 may be in response to a manual operation (e.g., button activation) or an automatic operation (e.g., puff-activation). In at least some example embodiments, the control circuity may include a haptic motor that may be disposed on a side of the power source 600.

The power source 600 may include one or more batteries (e.g., rechargeable dual battery arrangement, lithium-ion battery, fuel cells, or any combination thereof). It should be understood that the shape of the battery (or batteries) for the power supply 600 may vary. For example, the battery may be cylindrical, prismatic, disc-shaped, a pouch battery, or any other variation of battery shape known in the art. Additionally, it should be understood that the battery may be any of a variety of types. For example, in at least one example embodiment, the battery may include a rechargeable battery (e.g., lithium-ion). In another example embodiment, the battery may include a non-rechargeable battery (e.g., alkaline). In yet another example embodiment, the battery may include silver oxide, carbon zinc, cadmium, nickel, or any another material known in the art. Furthermore, the battery may include a primary cell and/or a secondary cell. It will be understood by those of ordinary skill in the art that various changes in form and details of the battery may be made without departing from the spirit and the scope of the invention.

The heater 402 may be electrically connected to the power source 600. In at least one example embodiment, the heater 402 may be configured to subject the consumable 200 to continuous heating or substantially continuous heating when the heater 402 is activated by supplying power from the power source 600 to the heater 402.

In at least one example embodiment, the aerosol-forming substrate 100 including the binder may be configured to facilitate sustained release of constituents (e.g., an active material such as nicotine). In at least one example embodiment, the aerosol-forming substrate 100 may be configured to be constantly heated for a series of about ten puffs to release a total amount of released constituent (e.g., nicotine). The aerosol-generating substrate 100 may be configured to release less than about 70 weight percent of the total amount of released constituent during a first five puffs of the series of ten puffs (e.g., less than or equal to about 65 weight percent, less than or equal to about 60 weight percent, less than or equal to about 57 weight percent, less than or equal to about 55 weight percent, less than or equal to about 53 weight percent, less than or equal to about 52 weight percent, or less than or equal to about 51 weight percent). In at least one example embodiment, the aerosol-forming substrate 100 may be configured to release constituent in an amount ranging from 35 weight percent of the total amount of released constituent during the first five puffs of the series of ten puffs (e.g., greater than or equal to about 43 weight percent, greater than or equal to about 45 weight percent, greater than or equal to about 48 weight percent, or greater than or equal to about 50 weight percent).

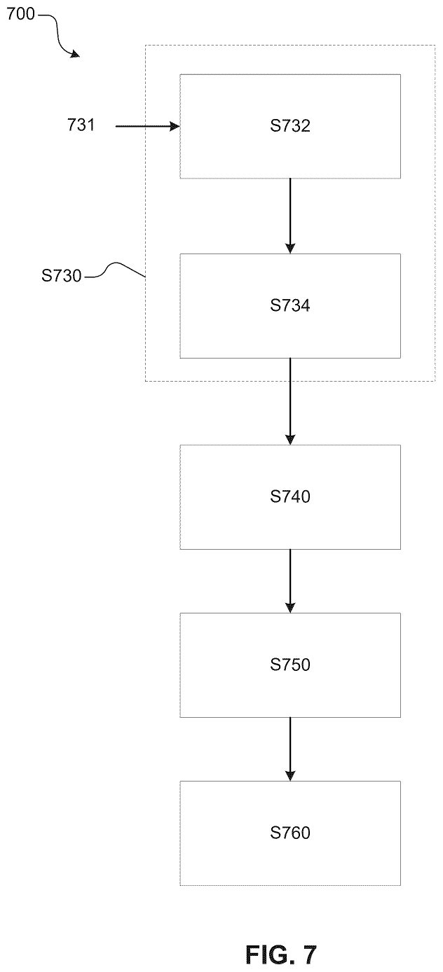

FIG. 7 is a flowchart illustrating an example method 700 of preparing an aerosol-forming substrate, like the aerosol-forming substrate illustrated in FIG. 1, according to at least one example embodiment.

The method 700 may include spheronizing 730 a wet mass to form a homogeneous or uniformed botanical granules that define, or are included in, the aerosol-forming substrate.

In at least one example embodiment, the wet mass may have a moisture level greater than or equal to about 35 % (e.g., greater than or equal to about 40 %, greater than or equal to about 45 %, greater than or equal to about 50 %, or greater than or equal to about 55 %). In at least one example embodiment, the wet mass may have a moisture level less than or equal to about 60 % (e.g., less than or equal to about 55 %, less than or equal to about 50 %, less than or equal to about 45 %, or less than or equal to about 40 %). In at least one example embodiment, the wet mass may have a moisture level greater than or equal to about 35 % to less than or equal to about 60 %.

The wet mass may include a botanical powder and a solvent. For example, in at least one example embodiment, the wet mass may include greater than or equal to about 40 weight percent (e.g., greater than or equal to about 45 weight percent, greater than or equal to about 50 weight percent, greater than or equal to about 55 weight percent, or greater than or equal to about 60 % weight percent) of the botanical powder. In at least one example embodiment, the wet mass may include less than or equal to about 65 weight percent (e.g., less than or equal to about 60 weight percent, less than or equal to about 55 weight percent, less than or equal to about 50 weight percent, or less than or equal to about 45 weight percent) of the botanical powder. In at least one example embodiment, the wet mass may include greater than or equal to about 40 weight percent to less than or equal to about 65 weight percent of the botanical powder.

In at least one example embodiment, the wet mass may include greater than or equal to about 35 weight percent (e.g., greater than or equal to about 40 weight percent, greater than or equal to about 45 weight percent, greater than or equal to about 50 weight percent, or greater than or equal to about 55 weight percent) of the solvent. In at least one example embodiment, the wet mass may include less than or equal to about 60 weight percent (e.g., less than or equal to about 55 weight percent, less than or equal to about 50 weight percent, less than or equal to about 45 weight percent, or less than or equal to about 40 weight percent) of the solvent. In at least one example embodiment, the wet mass may include greater than or equal to about 35 weight percent to less than or equal to about 60 weight percent of the solvent.

In at least one example embodiment, the botanical powder may include a plurality of botanical particles, where the botanical particles have average particle sizes of less than or equal to about 2 millimeters (e.g., less than or equal to about 1,900 micrometers, less than or equal to about 1,800 micrometers, less than or equal to about 1,700 micrometers, less than or equal to about 1,600 micrometers, less than or equal to about 1,500 micrometers, less than or equal to about 1,400 micrometers, less than or equal to about 1,300 micrometers, less than or equal to about 1,200 micrometers, less than or equal to about 1,100 micrometers, less than or equal to about 1,000 micrometers, less than or equal to about 900 micrometers, less than or equal to about 800 micrometers, less than or equal to about 700 micrometers, less than or equal to about 600 micrometers, or less than or equal to about 500 micrometers).

In at least one example embodiment, the botanical powder may include star anise, coco, fennel, eucalyptus, dill, cinnamon, fenugreek, ginger, basil, holy basil, caraway, rosemary, clove, rose petals, ginger, lavender, oak wood, or any combination thereof. In at least one example embodiment, the solvent may be an aqueous solvent.

The spheronizing S730 may include introducing S732 the wet mass 731 into a container (e.g., bowl) and applying S734 a centrifugal force to the wet mass 731. The botanical particles of the wet mass 731 are brought into continuous collisions with each other and also with the container and components thereof. In at least one example embodiment, the centrifugal force may be applied S734 after the wet mass 731 is introduced S732 into the container. In other example embodiments, the centrifugal force may be applied S734 as the wet mass 731 is introduced S732 into the container. In still other example embodiments, the centrifugal force may be applied S734 both as the wet mass 731 is introduced S732 into the container and after the wet mass 731 is introduced S732 into the container.

In at least one example embodiment, the container may be a stationary container including a movable element, such as a friction plate. In at least one example embodiment, the movable element may be rotated to apply S734 the centrifugal force. In at least one example embodiment, the movable element may be rotated at a centrifugal speed greater than or equal to about 600 rotations per minute (e.g., greater than or equal to about 700 rotations per minute, greater than or equal to about 800 rotations per minute, greater than or equal to about 900 rotations per minute, greater than or equal to about 1,000 rotations per minute, greater than or equal to about 1,100 rotations per minute, greater than or equal to about 1,200 rotations per minute, greater than or equal to about 1,300 rotations per minute, greater than or equal to about 1,400 rotations per minute, greater than or equal to about 1,500 rotations per minute, greater than or equal to about 1,600 rotations per minute, greater than or equal to about 1,700 rotations per minute, greater than or equal to about 1,800 rotations per minute, or greater than or equal to about 1,900 rotations per minute). In at least one example embodiment, the movable element may be rotated at a centrifugal speed less than or equal to about 2,000 rotations per minute (e.g., less than or equal to about 1,900 rotations per minute, less than or equal to about 1,800 rotations per minute, less than or equal to about 1,700 rotations per minute, less than or equal to about 1,600 rotations per minute, less than or equal to about 1,500 rotations per minute, less than or equal to about 1,400 rotations per minute, less than or equal to about 1,300 rotations per minute, less than or equal to about 1,200 rotations per minute, less than or equal to about 1,100 rotations per minute, less than or equal to about 1,000 rotations per minute, less than or equal to about 900 rotations per minute, less than or equal to about 800 rotations per minute, or less than or equal to about 700 rotations per minute). In at least one example embodiment, the movable element may be rotated at a centrifugal speed greater than or equal to about 600 rotations per minute to less than or equal to about 2,000 rotations per minute.

In at least one example embodiment, the container may be a stationary container including a movable element that may include one or more grooves that may help to enhance the frictional interactions between the botanical particles of the wet mass 731. In at least one example embodiment, the groove may have a groove size greater than or equal to about 2 millimeters (e.g., greater than or equal to about 2.1 millimeters, greater than or equal to about 2.2 millimeters, greater than or equal to about 2.3 millimeters, greater than or equal to about 2.4 millimeters, greater than or equal to about 2.5 millimeters, greater than or equal to about 2.6 millimeters, greater than or equal to about 2.7 millimeters, greater than or equal to about 2.8 millimeters, greater than or equal to about 2.9 millimeters, greater than or equal to about 3 millimeters, greater than or equal to about 3.1 millimeters, greater than or equal to about 3.2 millimeters, greater than or equal to about 3.3 millimeters, greater than or equal to about 3.4 millimeters, greater than or equal to about 3.5 millimeters, greater than or equal to about 3.6 millimeters, greater than or equal to about 3.7 millimeters, greater than or equal to about 3.8 millimeters, or greater than or equal to about 3.9 millimeters). In at least one example embodiment, the groove may have a groove size less than or equal to about 4 millimeters (e.g., less than or equal to about 3.9 millimeters, less than or equal to about 3.8 millimeters, less than or equal to about 3.7 millimeters, less than or equal to about 3.6 millimeters, less than or equal to about 3.5 millimeters, less than or equal to about 3.4 millimeters, less than or equal to about 3.3 millimeters, less than or equal to about 3.2 millimeters, less than or equal to about 3.1 millimeters, less than or equal to about 3 millimeters, less than or equal to about 2.9 millimeters, less than or equal to about 2.8 millimeters, less than or equal to about 2.7 millimeters, less than or equal to about 2.6 millimeters, less than or equal to about 2.5 millimeters, less than or equal to about 2.4 millimeters, less than or equal to about 2.3 millimeters, less than or equal to about 2.2 millimeters, or less than or equal to about 2.1 millimeters).

In at least one example embodiment, the wet mass may be included in, or defines, a granulation solution that is introduced S732 into the container. The granulation solution may include a binder, an active material, a humectant, a controlled delivery agent, an aerosol-forming agent, or any combination thereof.

In at least one example embodiment, the granulation solution may include greater than or equal to about 35 weight percent (e.g., greater than or equal to about 40 weight percent, greater than or equal to about 45 weight percent, greater than or equal to about 50 weight percent, or greater than or equal to about 55 weight percent) of the wet mass. In at least one example embodiment, the granulation solution may include less than or equal to about 60 weight percent (e.g., less than or equal to about 55 weight percent, less than or equal to about 50 weight percent, less than or equal to about 45 weight percent, or less than or equal to about 40 weight percent) of the wet mass. In at least one example embodiment, the granulation solution may include greater than or equal to about 35 weight percent to less than or equal to about 60 weight percent of the wet mass.

The binder may include, for example, hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof. In at least one example embodiment, the granulation solution may include greater than or equal to about 1.5 weight percent (e.g., greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent) of the binder. In at least one example embodiment, the granulation solution may include less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, or less than or equal to about 2 weight percent) of the binder. In at least one example embodiment, the granulation solution may include greater than or equal to about 1.5 weight percent to less than or equal to about 5 weight percent of the binder.

The active material may include, for example, nicotine. The nicotine may include free base nicotine, a nicotine salt, or a combination of the free base nicotine and the nicotine salt. The nicotine salt may include, for example, citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof. In at least one example embodiment, the granulation solution may include greater than or equal to 0 weight percent (e.g., greater than or equal to about 0.2 weight percent, greater than or equal to about 0.4 weight percent, greater than or equal to about 0.6 weight percent, greater than or equal to about 0.8 weight percent, or greater than or equal to about 1 weight percent of the active material. In at least one example embodiment, the granulation solution may include less than or equal to about 1.2 weight percent (e.g., less than or equal to about 1 weight percent, less than or equal to about 0.8 weight percent, less than or equal to about 0.6 weight percent, less than or equal to about 0.4 weight percent, or less than or equal to about 0.2 weight percent) of the active material. In at least one example embodiment, the granulation solution may include greater than or equal to 0 weight percent to less than or equal to about 1.2 weight percent of the active material.

The humectant may include, for example, 1,2-propylene glycol, 1,3-propylene glycol, 1,3-butane diol, glycerol, or any combination thereof. In at least one example embodiment, the granulation solution may include greater than or equal to 0 weight percent (e.g., greater than or equal to about 1 weight percent, greater than or equal to about 2 weight percent, greater than or equal to about 3 weight percent, greater than or equal to about 4 weight percent, greater than or equal to about 5 weight percent, greater than or equal to about 6 weight percent, greater than or equal to about 7 weight percent, greater than or equal to about 8 weight percent, or greater than or equal to about 9 weight percent) of the humectant. In at least one example embodiment, the granulation solution may include less than or equal to about 10 weight percent (e.g., less than or equal to about 9 weight percent, less than or equal to about 8 weight percent, less than or equal to about 7 weight percent, less than or equal to about 6 weight percent, less than or equal to about 5 weight percent, less than or equal to about 4 weight percent, less than or equal to about 3 weight percent, less than or equal to about 2 weight percent, or less than or equal to about 1 weight percent) of the humectant. In at least one example embodiment, the granulation solution may include greater than or equal to 0 weight percent to less than or equal to about 10 weight percent of the humectant.

The controlled delivery agent may include, for example, a binder. The binder may include, for example, hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof. In at least one example embodiment, the granulation solution may include greater than or equal to about 1.5 weight percent (e.g., greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent) of the controlled delivery agent. In at least one example embodiment, the granulation solution may include less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, or less than or equal to about 2 weight percent) of the controlled delivery agent. In at least one example embodiment, the granulation solution may include greater than or equal to about 1.5 weight percent to less than or equal to about 5 weight percent of the controlled delivery agent.

The aerosol-forming agent may include, for example, 1,2-propylene glycol, 1,3-propylene glycol, 1,3-butane diol, glycerol, or any combination thereof. In at least one example embodiment, the granulation solution may include greater than or equal to about greater than or equal to 0 weight percent (e.g., greater than or equal to about 1 weight percent, greater than or equal to about 2 weight percent, greater than or equal to about 3 weight percent, greater than or equal to about 4 weight percent, greater than or equal to about 5 weight percent, greater than or equal to about 6 weight percent, greater than or equal to about 7 weight percent, greater than or equal to about 8 weight percent, or greater than or equal to about 9 weight percent) of the aerosol-forming agent. In at least one example embodiment, the granulation solution may include less than or equal to about 10 weight percent (e.g., less than or equal to about 9 weight percent, less than or equal to about 8 weight percent, less than or equal to about 7 weight percent, less than or equal to about 6 weight percent, less than or equal to about 5 weight percent, less than or equal to about 4 weight percent, less than or equal to about 3 weight percent, less than or equal to about 2 weight percent, or less than or equal to about 1 weight percent) of the aerosol-forming agent. In at least one example embodiment, the granulation solution may include greater than or equal to about greater than or equal to 0 weight percent to less than or equal to about 10 weight percent of the aerosol-forming agent.

The method 700 may include drying S740 the botanical granules to remove the solvent. The drying S740 may include applying a desired (or alternatively, predetermined) heat to the botanical granules. For example, in at least one example embodiment, the drying S740 may include placing the botanical granules in an oven at a desired (or alternatively, predetermined) temperature for a desired (or alternatively, predetermined) duration. In other example embodiments, the drying S740 may include fluidizing the botanical granules in a fluidized bed vessel at a desired (or alternatively, predetermined) temperature for a desired (or alternatively, predetermined) duration. For example, the drying S740 may include transferring the botanical granules to the fluidized bed.

In at least one example embodiment, the drying S740 may include applying a heat to the botanical granules greater than or equal to about 25° C. (e.g., greater than or equal to about 30° C., greater than or equal to about 35° C., greater than or equal to about 40° C., greater than or equal to about 45° C., greater than or equal to about 50° C., or greater than or equal to about 55° C.). In at least one example embodiment, the drying may include applying a heat to the botanical granules less than or equal to about 60° C. (e.g., less than or equal to about 55° C., less than or equal to about 50° C., less than or equal to about 45° C., less than or equal to about 40° C., less than or equal to about 35° C., or less than or equal to about 30° C.). In at least one example embodiment, the drying S740 may include applying a heat to the botanical granules greater than or equal to about 25° C. to less than or equal to about 60° C.

In at least one example embodiment, the drying S740 may include applying a heat to the botanical granules for a predetermined or selected time period. In at least one example embodiment, the drying S740 may include applying a heat to the botanical granules for a time period greater than or equal to about 10 minutes (e.g., greater than or equal to about 12 minutes, greater than or equal to about 14 minutes, greater than or equal to about 16 minutes, greater than or equal to about 18 minutes, greater than or equal to about 20 minutes, greater than or equal to about 22 minutes, greater than or equal to about 24 minutes, greater than or equal to about 26 minutes, or greater than or equal to about 28 minutes). In at least one example embodiment, the drying S740 may include applying a heat to the botanical granules for a time period less than or equal to about 30 minutes (e.g., less than or equal to about 28 minutes, less than or equal to about 26 minutes, less than or equal to about 24 minutes, less than or equal to about 22 minutes, less than or equal to about 20 minutes, less than or equal to about 18 minutes, less than or equal to about 16 minutes, less than or equal to about 14 minutes, or less than or equal to about 12 minutes). In at least one example embodiment, the drying S740 may include applying a heat to the botanical granules for a time period greater than or equal to about 10 minutes to less than or equal to about 30 minutes.

The method 700 may include preparing S750 an aerosol-forming substrate from the botanical granules or the dried botanical granules. In at least one example embodiment, the preparing S750 of the aerosol-forming substrate may be prepared by applying a mixing force.

The method 700 may include preparing S760 a consumable including the aerosol-forming substrate. In at least one example embodiment, the preparing S760 of the consumable may include adding the aerosol-forming substrate to an annular space of a container (such as the substrate region 316 as illustrated in FIG. 3).

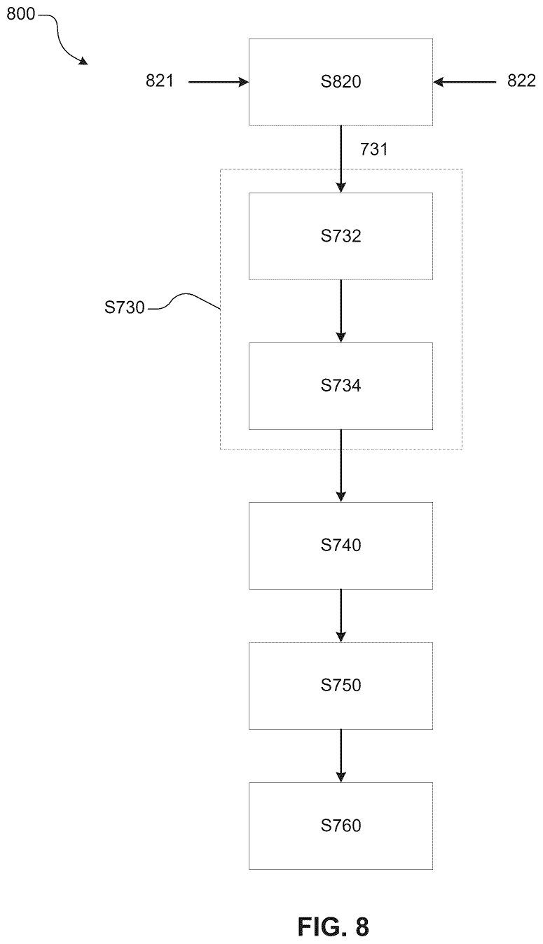

FIG. 8 is a flowchart illustrating another example method 800 of preparing an aerosol-forming substrate, like the aerosol-forming substrate illustrated in FIG. 1, according to at least one example embodiment.

The method 800 as illustrated in FIG. 8 may be the same as the method 700 illustrated in FIG. 7 except that the method 800 includes preparing S820 the wet mass 731. In at least one example embodiment, preparing S820 the wet mass 731 may include contacting the botanical powder 821 to the solvent 822. The contacting may include adding the solvent 822 to the botanical powder 821 in two or more increments. The contacting may include applying a mixing force to the botanical powder 821 before, during, or after the addition of the solvent.

The mixing force may be applied for a desired (or alternatively, predetermined) time period. In at least one example embodiment, the mixing force may be applied for a time period greater than or equal to about 5 minutes (e.g., greater than or equal to about 6 minutes, greater than or equal to about 7 minutes, greater than or equal to about 8 minutes, greater than or equal to about 9 minutes, greater than or equal to about 10 minutes, greater than or equal to about 11 minutes, greater than or equal to about 12 minutes, greater than or equal to about 13 minutes, or greater than or equal to about 14 minutes). In at least one example embodiment, the mixing force may be applied for a time period less than or equal to about 15 minutes (e.g., less than or equal to about 14 minutes, less than or equal to about 13 minutes, less than or equal to about 12 minutes, less than or equal to about 11 minutes, less than or equal to about 10 minutes, less than or equal to about 9 minutes, less than or equal to about 8 minutes, less than or equal to about 7 minutes, or less than or equal to about 6 minutes). In at least one example embodiment, the mixing force may be applied for a time period greater than or equal to about 5 minutes to less than or equal to about 15 minutes.

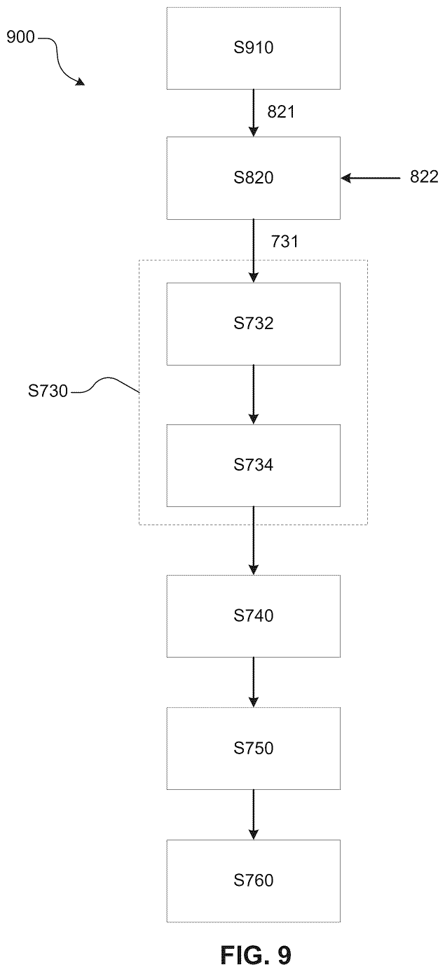

FIG. 9 is a flowchart illustrating another example method 900 of preparing an aerosol-forming substrate, like the aerosol-forming substrate illustrated in FIG. 1, according to at least one example embodiment.

The method 900 as illustrated in FIG. 9 may be the same as the method 700 illustrated in FIG. 7 and/or the method 800 as illustrated in FIG. 8 except that the method 900 includes isolating S810 the botanical particles having the average particle sizes of less than or equal to about 2 millimeters. In at least one example embodiment, the isolating may include moving a precursor botanical powder through a mesh. In at least one example embodiment, the mesh may have an average pore size of about 2 millimeters.

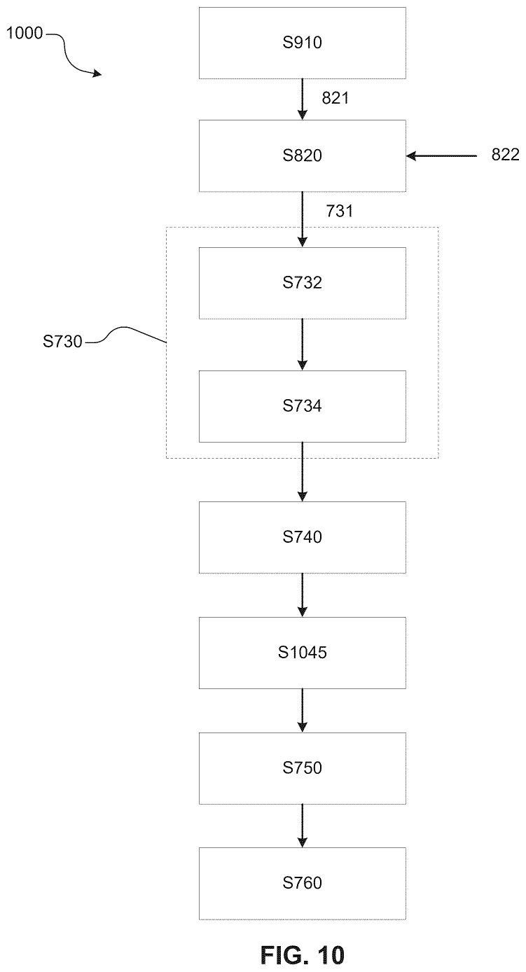

FIG. 10 is a flowchart illustrating another example method 1000 of preparing an aerosol-forming substrate, like the aerosol-forming substrate illustrated in FIG. 1, according to at least one example embodiment.

The method 1000 as illustrated in FIG. 10 may be the same as the method 700 illustrated in FIG. 7 and/or the method 800 as illustrated in FIG. 8 and/or the method 900 as illustrated in FIG. 9 except that the method 1000 includes, before the preparing S750 of the aerosol-forming substrate from the botanical granules or the dried botanical granules and/or the preparing S760 of the consumable, sieving S1045 the botanical granules or the dried botanical granules to confirm a particle sizes of the botanical granules or the dried botanical granules. In at least one example embodiment, the sieving S1045 may include moving the botanical granules or the dried botanical granules through a mesh having an average pore size greater than a size 10 mesh. In at least one example embodiment, the sieving S1045 may include moving the botanical granules or the dried botanical granules through a mesh having an average pore size less than a size 20 mesh.

While some example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

Although described with reference to specific examples and drawings, modifications, additions and substitutions of example embodiments may be variously made according to the description by those of ordinary skill in the art. For example, the described techniques may be performed in an order different with that of the methods described, and/or elements such as the described system, architecture, devices, circuit, and the like, may be connected or combined to be different from the above-described methods, or results may be appropriately achieved by other elements or equivalents.

Illustrative embodiment 1. A method for preparing botanical granules for use in heated inhalable products, the method comprising: spheronizing a wet mass to form the botanical granules, the wet mass including a botanical powder and a solvent, the botanical powder including botanical particles having average particle sizes of less than or equal to about 2 millimeters.

Illustrative embodiment 2. The method of illustrative embodiment 1, further comprising: contacting the botanical powder with the solvent to form the wet mass.

Illustrative embodiment 3. The method of illustrative embodiment 2, wherein the contacting includes adding the solvent to the botanical powder in increments.

Illustrative embodiment 4. The method of any of illustrative embodiments 2 and 3, wherein the contacting includes applying a mixing force to the botanical powder.

Illustrative embodiment 5. The method of illustrative embodiment 4, wherein the mixing force is applied for a time period less than or equal to about 15 minutes.

Illustrative embodiment 6. The method of any of illustrative embodiments 4 and 5, wherein the mixing force is applied for a time period greater than or equal to about 5 minutes.

Illustrative embodiment 7. The method of any of illustrative embodiments 2-6, further comprising: before the contacting, isolating the botanical particles having the average particle sizes of less than or equal to 2 millimeters.

Illustrative embodiment 8. The method of illustrative embodiment 7, wherein the isolating includes moving precursor botanical particles through a mesh.

Illustrative embodiment 9. The method of any of illustrative embodiments 1-8, wherein the solvent is an aqueous solvent.

Illustrative embodiment 10. The method of any of illustrative embodiments 1-9, wherein the wet mass are included in a granulation solution.

Illustrative embodiment 11. The method of illustrative embodiment 10, wherein the granulation solution further comprises: a binder, an active material, a humectant, or any combination thereof.

Illustrative embodiment 12. The method of illustrative embodiment 11, wherein the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

Illustrative embodiment 13. The method of any of illustrative embodiments 11 and 12, wherein the active material includes nicotine.

Illustrative embodiment 14. The method of illustrative embodiment 13, wherein the nicotine includes free base nicotine, a nicotine salt, or a combination of the free base nicotine and the nicotine salt.

Illustrative embodiment 15. The method of illustrative embodiment 14, wherein the nicotine salt includes citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof.

Illustrative embodiment 16. The method of any of illustrative embodiments 11-15, wherein the humectant includes 1,2-propylene glycol, 1,3-propylene glycol, 1,3-butane diol, glycerol, or any combination thereof.

Illustrative embodiment 17. The method of any of illustrative embodiments 11-16,further comprising: contacting the binder, the active material, the humectant, or the combination thereof with the wet mass to form the granulation solution.

Illustrative embodiment 18. The method of any of illustrative embodiments 10-17, wherein the granulation solution further comprises: a controlled delivery agent.

Illustrative embodiment 19. The method of illustrative embodiment 18, wherein the controlled delivery agent includes a binder.

Illustrative embodiment 20. The method of illustrative embodiment 19, wherein the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

Illustrative embodiment 21. The method of any of illustrative embodiments 18-20, further comprising: contacting the controlled delivery agent with the wet mass to form the granulation solution.

Illustrative embodiment 22. The method of any of illustrative embodiments 10-21, wherein the granulation solution further includes an aerosol-forming agent.

Illustrative embodiment 23. The method of illustrative embodiment 22, wherein the aerosol-forming agent includes 1,2-propylene glycol, 1,3-propylene glycol, 1,3-butane diol, glycerol, or any combination thereof.

Illustrative embodiment 24. The method of any of illustrative embodiments 22 and 23, further comprising: contacting the aerosol-forming agent with the wet mass to form the granulation solution.

Illustrative embodiment 25. The method of any of illustrative embodiments 1-24, wherein the spheronizing includes: introducing the wet mass into a bowl; and applying a centrifugal force.

Illustrative embodiment 26. The method of illustrative embodiment 25, wherein the bowl is a stationary bowl, and the stationary bowl includes a friction plate.

Illustrative embodiment 27. The method of illustrative embodiment 26, wherein a centrifugal speed of the friction plate is greater than or equal to about 600 rotations per minute.

Illustrative embodiment 28. The method of any of illustrative embodiments 26 and 27, wherein a centrifugal speed of the friction plate is less than or equal to about 2,000 rotations per minute.

Illustrative embodiment 29. The method of any of illustrative embodiments 26-28, wherein a centrifugal speed of the friction plate is about 1,500 rotations per minute.

Illustrative embodiment 30. The method of any of illustrative embodiments 26-29, wherein the friction plate has a groove.

Illustrative embodiment 31. The method of illustrative embodiment 30, wherein the groove has a groove size greater than or equal to about 2 millimeters.

Illustrative embodiment 32. The method of any of illustrative embodiments 30 and 31, wherein the groove has a groove size less than or equal to about 4 millimeters.

Illustrative embodiment 33. The method of any of illustrative embodiments 1-32, wherein the botanical granules have an average particle size greater than or equal to 500 micrometers.

Illustrative embodiment 34. The method of any of illustrative embodiments 1-33, wherein the botanical granules have an average particle size less than or equal to about 2,500 micrometers.

Illustrative embodiment 35. The method of any of illustrative embodiments 1-34, wherein the botanical granules have a bulk density greater than or equal to about 0.23 g/cc.

Illustrative embodiment 36. The method of any of illustrative embodiments 1-35, wherein the botanical granules have a bulk density less than or equal to about 0.25 g/cc.

Illustrative embodiment 37. The method of any of illustrative embodiments 1-36, wherein the botanical granules have a tapped density greater than or equal to about 0.3 g/cc.

Illustrative embodiment 38. The method of any of illustrative embodiments 1-37, wherein the botanical granules have a tapped density less than or equal to about 0.32 g/cc.

Illustrative embodiment 39. The method of any of illustrative embodiments 1-38, further comprising: drying the botanical granules to remove the solvent.

Illustrative embodiment 40. The method of illustrative embodiment 39, wherein the drying includes transferring the botanical granules to a fluidized bed.

Illustrative embodiment 41. The method of any of illustrative embodiments 1-40, further comprising: sieving the botanical granules to confirm particle size.

Illustrative embodiment 42. The method of any of any of illustrative embodiments 1-41, wherein the botanical powder includes star anise, coco, fennel, eucalyptus, dill, cinnamon, fenugreek, ginger, basil, holy basil, caraway, rosemary, clove, rose petals, ginger, lavender, oak wood, or any combination thereof.

Illustrative embodiment 43. A method for preparing botanical granules for use in heated inhalable products, the method comprising: preparing a granulation solution, the granulation solution including a botanical powder and a solvent, the botanical powder including botanical particles having average particle sizes of less than or equal or equal to about 2 millimeters; and spheronizing the granulation solution.

Illustrative embodiment 44. The method of illustrative embodiment 43, wherein the preparation of the granulation solution includes contacting the botanical powder to the solvent.

Illustrative embodiment 45. The method of illustrative embodiment 44, wherein the contacting includes adding the solvent to the botanical powder in increments.

Illustrative embodiment 46. The method of any of illustrative embodiments 44 and 45, wherein the contacting includes applying a mixing force to the botanical powder.

Illustrative embodiment 47. The method of illustrative embodiment 46, wherein the mixing force is applied for a time period greater than or equal to about 5 minutes to less than or equal to about 15 minutes.

Illustrative embodiment 48. The method of any of illustrative embodiments 43-47, further comprising before the preparation of the granulation solution, isolating the botanical particles having the average particle sizes of less than or equal to 2 millimeters.

Illustrative embodiment 49. The method of illustrative embodiment 48, wherein the isolating includes moving precursor botanical particles through a mesh.

Illustrative embodiment 50. The method of any of illustrative embodiments 43-49, wherein the solvent is an aqueous solvent.

Illustrative embodiment 51. The method of any of illustrative embodiments 43-50, wherein the granulation solution further comprises: a binder, an active material, a humectant, or any combination thereof.

Illustrative embodiment 52. The method of illustrative embodiment 51, wherein the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

Illustrative embodiment 53. The method of any of illustrative embodiments 51 and 53, wherein the active material includes nicotine.

Illustrative embodiment 54. The method of illustrative embodiment 53, wherein the nicotine includes free base nicotine, a nicotine salt, or a combination of the free base nicotine and the nicotine salt.

Illustrative embodiment 55. The method of illustrative embodiment 54, wherein the nicotine salt includes citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof.

Illustrative embodiment 56. The method of any of illustrative embodiments 51-55, wherein the humectant includes 1,2-propylene glycol, 1,3-propylene glycol, 1,3-butane diol, glycerol, or any combination thereof.

Illustrative embodiment 57. The method of any of illustrative embodiments 43-56, wherein the granulation solution further comprises: a controlled delivery agent.

Illustrative embodiment 58. The method of illustrative embodiment 57, wherein the controlled delivery agent includes a binder.

Illustrative embodiment 59. The method of illustrative embodiment 58, wherein the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

Illustrative embodiment 60. The method of any of illustrative embodiments 43-59, wherein the granulation solution further includes an aerosol-forming agent.

Illustrative embodiment 61. The method of illustrative embodiment 60, wherein the aerosol-forming agent includes 1,2-propylene glycol, 1,3-propylene glycol, 1,3-butane diol, glycerol, or any combination thereof.

Illustrative embodiment 62. The method of any of illustrative embodiments 43-61, wherein the spheronizing includes: introducing the wet mass into a bowl; and applying a centrifugal force.

Illustrative embodiment 63. The method of illustrative embodiment 62, wherein the bowl is a stationary bowl, and the stationary bowl includes a friction plate.

Illustrative embodiment 64. The method of illustrative embodiment 63, wherein a centrifugal speed of the friction plate is greater than or equal to about 600 rotations per minute to less than or equal to about 2,000 rotations per minute.

Illustrative embodiment 65. The method of any of illustrative embodiments 63 and 64, wherein the friction plate has a groove.

Illustrative embodiment 66. The method of illustrative embodiment 65, wherein the groove has a groove size greater than or equal to about 2 millimeters to less than or equal to about 4 millimeters.

Illustrative embodiment 67. The method of any of illustrative embodiments 43-66, wherein the botanical granules have an average particle size greater than or equal to 500 micrometers to less than or equal to about 2,500 micrometers.

Illustrative embodiment 68. The method of any of illustrative embodiments 43-67, wherein the botanical granules have a bulk density greater than or equal to about 0.23 g/cc to less than or equal to about 0.25 g/cc.

Illustrative embodiment 69. The method of any of illustrative embodiments 43-68, wherein the botanical granules have a tapped density greater than or equal to about 0.3 g/cc to less than or equal to about 0.32 g/cc.

Illustrative embodiment 70. The method of any of illustrative embodiments 43-69, further comprising: drying the botanical granules to remove the solvent.

Illustrative embodiment 71. The method of illustrative embodiment 70, wherein the drying includes transferring the botanical granules to a fluidized bed.

Illustrative embodiment 72. The method of any of illustrative embodiments 43-71, further comprising: sieving the botanical granules to confirm particle size.

Illustrative embodiment 73. The method of any of illustrative embodiments 43-72, wherein the botanical powder includes star anise, coco, fennel, eucalyptus, dill, cinnamon, fenugreek, ginger, basil, holy basil, caraway, rosemary, clove, rose petals, ginger, lavender, oak wood, or any combination thereof.

Illustrative embodiment 74. A heated inhalable product comprising: a device body including: a housing defining a chamber, a heater, and a power supply configured to supply power to the heater; and a consumable configured to be at least partially received in the chamber, the consumable including a container defining an annular interior region, an outer surface of the container configured to be heated by the heater; and a filler in the annular interior region, the filler including substantially homogeneous botanical particles.

Illustrative embodiment 75. The heated inhalable product of illustrative embodiment 74, wherein the annular interior region further includes a binder, an active material, a humectant, or any combination thereof.

Illustrative embodiment 76. The heated inhalable product of illustrative embodiment 75, wherein the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

Illustrative embodiment 77. The heated inhalable product of any of illustrative embodiments 75 and 76, wherein the active material includes nicotine.

Illustrative embodiment 78. The heated inhalable product of illustrative embodiment 77, wherein the nicotine includes free base nicotine, a nicotine salt, or a combination of the free base nicotine and the nicotine salt.

Illustrative embodiment 79. The heated inhalable product of illustrative embodiment 78, wherein the nicotine salt includes citrate, monotartrate, bitartrate, bitartrate dihydrate, salicylate, sulfate, bisulfate, phosphate, acid phosphate, acetate, lactate, succinate, maleate, fumarate, gluconate, saccharate, benzoate, methanesulfonate, hydrochloride, hydrobromide, hydroiodide, or any combination thereof.

Illustrative embodiment 80. The heated inhalable product of any of illustrative embodiments 75-79, wherein the humectant includes 1,2-propylene glycol, 1,3-propylene glycol, 1,3-butane diol, glycerol, or any combination thereof.

Illustrative embodiment 81. The heated inhalable product of any of illustrative embodiments 74-80, wherein the annular interior region further includes a controlled delivery agent.

Illustrative embodiment 82. The heated inhalable product of illustrative embodiment 81, wherein the controlled delivery agent includes a binder.

Illustrative embodiment 83. The heated inhalable product of illustrative embodiment 82, wherein the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof.

Illustrative embodiment 84. The heated inhalable product of any of illustrative embodiments 74-83, wherein the annular interior region further includes an aerosol-forming agent.

Illustrative embodiment 85. The heated inhalable product of illustrative embodiment 84, wherein the aerosol-forming agent includes 1,2-propylene glycol, 1,3-propylene glycol, 1,3-butane diol, glycerol, or any combination thereof.

Illustrative embodiment 86. The heated inhalable product of any of illustrative embodiments 74-85, wherein the botanical particles are prepared by spheronizing a wet mass including precursor botanical particles, the precursor botanical particles having average particle sizes of less than or equal to about 2 millimeters.

Illustrative embodiment 87. A method for preparing botanical granules for use in heated inhalable products, the method comprising: spheronizing a wet mass to form the botanical granules, the wet mass including a botanical powder and a solvent, the botanical powder including botanical particles having average particle sizes of less than or equal to about 2 millimeters.

Illustrative embodiment 88. The method of illustrative embodiment 87, further comprising: contacting the botanical powder with the solvent to form the wet mass, the contacting including at least one of adding the solvent to the botanical powder in increments and applying a mixing force to the botanical powder.

Illustrative embodiment 89. The method of illustrative embodiment 88, further comprising: before the contacting, isolating the botanical particles having the average particle sizes of less than or equal to 2 millimeters.

Illustrative embodiment 90. The method of any of illustrative embodiments 87-89, wherein the solvent is an aqueous solvent.

Illustrative embodiment 91. The method of any of illustrative embodiments 87-90, wherein the wet mass are included in a granulation solution, the granulation solution further including a binder, an active material, a humectant, or any combination thereof.

Illustrative embodiment 92. The method of illustrative embodiment 91, wherein the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof; the active material includes nicotine; the humectant includes 1,2-propylene glycol, 1,3-propylene glycol, 1,3-butane diol, glycerol, or any combination thereof.

Illustrative embodiment 93. The method of any of illustrative embodiments 91 and 92, further comprising: contacting the binder, the active material, the humectant, or the combination thereof with the wet mass to form the granulation solution.

Illustrative embodiment 94. The method of any of illustrative embodiments 91-93, wherein the granulation solution further includes an aerosol-forming agent.

Illustrative embodiment 95. The method of illustrative embodiment 94, wherein the aerosol-forming agent includes 1,2-propylene glycol, 1,3-propylene glycol, 1,3-butane diol, glycerol, or any combination thereof.

Illustrative embodiment 96. The method of any of illustrative embodiments 94 and 95, further comprising: contacting the aerosol-forming agent with the wet mass to form the granulation solution.

Illustrative embodiment 97. The method of any of illustrative embodiment 87-96, wherein the spheronizing includes introducing the wet mass into a stationary bowl including a friction plate; and applying a centrifugal force.

Illustrative embodiment 98. The method of illustrative embodiment 97, wherein a centrifugal speed of the friction plate is greater than or equal to about 600 rotations per minute to less than or equal to about 2,000 rotations per minute.

Illustrative embodiment 99. The method of any of illustrative embodiments 97 and 98, wherein the friction plate has a groove.

Illustrative embodiment 100. The method of illustrative embodiment 99, wherein the groove has a groove size greater than or equal to about 2 millimeters to less than or equal to about 4 millimeters.

Illustrative embodiment 101. The method of any of illustrative embodiments 87-100, wherein the botanical granules have an average particle size greater than or equal to 500 micrometers to less than or equal to about 2,500 micrometers, the botanical granules have a bulk density greater than or equal to about 0.23 g/cc to less than or equal to about 0.25 g/cc, and the botanical granules have a tapped density greater than or equal to about 0.3 g/cc to less than or equal to about 0.32 g/cc.

Illustrative embodiment 102. The method of any of illustrative embodiments 87-101, further comprising: drying the botanical granules to remove the solvent by transferring the botanical granules to a fluidized bed.

Illustrative embodiment 103. The method of any of illustrative embodiments87-103, further comprising: sieving the botanical granules to confirm particle size.

Illustrative embodiment 104. A method for preparing botanical granules for use in heated inhalable products, the method comprising: preparing a granulation solution, the granulation solution including a botanical powder and a solvent, the botanical powder including botanical particles having average particle sizes of less than or equal or equal to about 2 millimeters; and spheronizing the granulation solution.

Illustrative embodiment 105. The method of illustrative embodiment 105, wherein the preparation of the granulation solution includes contacting the botanical powder to the solvent, the contacting including at least one of adding the solvent to the botanical powder in increments and applying a mixing force to the botanical powder.

Illustrative embodiment 106. The method of any of illustrative embodiments 105 and 106, wherein the spheronizing includes: introducing the granulation solution into a stationary bowl including a friction plate; and applying a centrifugal force.

What is claimed is:

1. A method for preparing botanical granules for use in heated inhalable products, the method comprising:
spheronizing a wet mass to form the botanical granules, the wet mass including a botanical powder and a solvent, the botanical powder including botanical particles having average particle sizes of less than or equal to about 2 millimeters.

2. The method of claim 1, further comprising:
contacting the botanical powder with the solvent to form the wet mass, the contacting including at least one of adding the solvent to the botanical powder in increments and applying a mixing force to the botanical powder.

3. The method of claim 2, further comprising:
before the contacting, isolating the botanical particles having the average particle sizes of less than or equal to 2 millimeters.

4. The method of claim 1, wherein the solvent is an aqueous solvent.

5. The method of claim 1, wherein the wet mass are included in a granulation solution, the granulation solution further including a binder, an active material, a humectant, or any combination thereof.

6. The method of claim 5, wherein
the binder includes hydroxypropyl methylcellulose (HPMC), sodium carboxymethylcellulose (NaCMC), polysaccharides cellulose, pectin, agar, locust bean gum, guar gum, gum tragacanth, agar, chitosan, xanthan gum, carrageenan, alginates, hydroxypropyl cellulose (HPC), microcrystalline cellulose (MCC), methyl cellulose (MC), ethyl cellulose (EC), or any combination thereof;
the active material includes nicotine;
the humectant includes 1,2-propylene glycol, 1,3-propylene glycol, 1,3-butane diol, glycerol, or any combination thereof.

7. The method of claim 5, further comprising:
contacting the binder, the active material, the humectant, or the combination thereof with the wet mass to form the granulation solution.

8. The method of claim 5, wherein the granulation solution further includes an aerosol-forming agent.

9. The method of claim 8, wherein the aerosol-forming agent includes 1,2-propylene glycol, 1,3-propylene glycol, 1,3-butane diol, glycerol, or any combination thereof.

10. The method of claim 8, further comprising:
contacting the aerosol-forming agent with the wet mass to form the granulation solution.

11. The method of claim 1, wherein the spheronizing includes introducing the wet mass into a stationary bowl including a friction plate; and
applying a centrifugal force.

12. The method of claim 11, wherein a centrifugal speed of the friction plate is greater than or equal to about 600 rotations per minute to less than or equal to about 2,000 rotations per minute.

13. The method of claim 11, wherein the friction plate has a groove.

14. The method of claim 13, wherein the groove has a groove size greater than or equal to about 2 millimeters to less than or equal to about 4 millimeters.

15. The method of claim 1, wherein
the botanical granules have an average particle size greater than or equal to 500 micrometers to less than or equal to about 2,500 micrometers,
the botanical granules have a bulk density greater than or equal to about 0.23 g/cc to less than or equal to about 0.25 g/cc, and
the botanical granules have a tapped density greater than or equal to about 0.3 g/cc to less than or equal to about 0.32 g/cc.

16. The method of claim 1, further comprising:
drying the botanical granules to remove the solvent by transferring the botanical granules to a fluidized bed.

17. The method of claim 1, further comprising:
sieving the botanical granules to confirm particle size.

18. A method for preparing botanical granules for use in heated inhalable products, the method comprising:
preparing a granulation solution, the granulation solution including a botanical powder and a solvent, the botanical powder including botanical particles having average particle sizes of less than or equal or equal to about 2 millimeters; and
spheronizing the granulation solution.

19. The method of claim 18, wherein the preparation of the granulation solution includes contacting the botanical powder to the solvent, the contacting including at least one of adding the solvent to the botanical powder in increments and applying a mixing force to the botanical powder.

20. The method of claim 18, wherein the spheronizing includes:
introducing the granulation solution into a stationary bowl including a friction plate; and
applying a centrifugal force.
